(19) United States
(12) Patent Application Publication
(10) Pub. No.: US 2008/0103359 A1
(43) Pub. Date: May 1, 2008

(54) Capsule-type endoscopic system with real-time image display

(76) Inventor: Tah-Yoong Lin, Longtan Township (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/586,534

(22) Filed: Oct. 26, 2006

(51) Int. Cl. A61B 1/04; A61B 1/06

(52) U.S. Cl. 600/109; 600/118; 600/160

(57) ABSTRACT

The present invention relates to a capsule-type endoscopic system with real-time image display, which includes a capsule-type endoscope and a display apparatus. The capsule-type endoscope extracts an endoscopic image inside of a human body, produces endoscopic image data, and transmits the endoscopic image data to the display apparatus. The display apparatus displays immediately the endoscopic image data. For patients suffering from digestive diseases, physicians can acquire real-time endoscopic images through the capsule-type endoscopic system according to the present invention. If, according to the real-time endoscopic images of the present invention, the patients suffer from digestive diseases that need emergent treatment, the physicians can perform treatments immediately to the patients. Thereby, the diseases can be cured at early stage, and deterioration of emergent and lethal digestive diseases due to delay in treatment can be avoided.

### FIELD OF THE INVENTION

The present invention relates to an endoscopic system, and particularly to a capsule-type endoscopic system with real-time image display.

### BACKGROUND OF THE INVENTION

The capsule-type endoscope is a newly developed endoscope technology. The capsule-type endoscopic system accompanying the capsule-type endoscope can observe carefully diseases in the alimentary canal of a human body, especially diseases happening in the small intestines. Physicians can observe directly the conditions of the small intestines by means of the capsule-type endoscopic system to diagnose patients' conditions, and thereby the effects of medical examinations can be enhanced. A capsule endoscopic system according to the prior art is disclosed in the Taiwan Patent Number 1235051 entitled “Real-Time Capsule Endoscopic System”. FIG. 1 shows a block diagram of the capsule-type endoscopic system according to the prior art. As shown in FIG. 1, the capsule-type endoscopic system according to the prior art includes a capsule-type endoscope 10, a data recorder 20, and an image processor 30. The capsule-type endoscope 10 includes a camera 12, a wireless transmitting unit 14, and a battery 16. The shape and size of the capsule-type endoscope 10 are similar to those of a medical capsule for easier swallowing by patients under endoscopy. The capsule-type endoscope 10 extracts the endoscopic images of the alimentary canal by means of the camera 12, which will thereby produce corresponding endoscopic image data according to the extracted endoscopic images.

The data recorder 20 includes a wireless receiving unit 22 and a memory 24. The wireless receiving unit 22 connects to the wireless transmitting unit 14 via a wireless communication interface. The endoscopic images produced by the capsule-type endoscope 10 are transmitted to the wireless receiving unit 22 from the wireless transmitting unit 14 for storing the endoscopic image data received by the wireless receiving unit 22 to the memory 24. The image processor 30 couples to a display 32. When the data recorder 20 couples to the image processor 30, the image processor 30 will read the endoscopic image data stored in the memory 24, and will output the read endoscopic image data to the display 32 for displaying the endoscopic image data on the display 32. When a patient starts to accept endoscopy, the patient first swallows the capsule-type endoscope 10 and attaches the data recorder to his waist. After the patient swallowing the capsule-type endoscope 10, the capsule-type endoscope 10 will produce endoscopic image data according to endoscopic images of the alimentary canal extracted by the camera 12, and will transmit the endoscopic image data to the wireless receiving unit 22 via the wireless transmitting unit 14. The data recorder 20 will store the endoscopic image data received by the wireless receiving unit 22 until the power of the battery 16 carried by the capsule-type endoscope 10 is exhausted.

It takes about several hours from start for the capsule-type endoscope 10 to exhaust the power of the battery 16. During the period, the capsule-type endoscope 10 takes the endoscopic images of the stomach and the small intestine while passing them by means of peristalsis of the alimentary canal. Next, physicians read the endoscopic image data stored in the data recorder 20 by using the image processor 30, and display the endoscopic image data via the display 32 coupled to the image processor 30. Thereby, physicians can examine the alimentary canal of patients as a reference for diagnosis. If a patient suffers from a digestive disease which needs emergent treatment, the physician hopes to see the images of the alimentary canal of the patient extracted by the capsule-type endoscope 10 immediately, thereby he can judge the disease suffered by the patient at once and perform necessary treatment. However, it takes eight hours before the data recorder 20 can be read. Hence, the digestive disease that needs emergent treatment cannot be handled at earliest stage, and the patient will lose opportunities of early diagnosis and treatment. In addition, because the data recorder 20 has to be hung around the patient's waist, it is necessary to consider the influence of the shape of the receiving antenna in the wireless receiving unit 22 of the data recorder on normal life of the patient. Thereby, the antenna of the wireless receiving unit 22 needs to be shrunk in volume and to be attached to the waist of the patient. Hence, the data recorder 20 is easily worn by the patient and the endoscopic image data transmitted by the capsule-type endoscope 10 can be stored therein. Besides, the display 32 needs to couple to the data recorder 20 through the image processor 30 and then read the endoscopic image data stored in the data recorder 20 for displaying the endoscopic image data. Thereby, the display cannot display the endoscopic image data real-timely.

Accordingly, a novel capsule-type endoscopic system, which is capable of solving the drawback of being unable to display image data real-timely and of enhancing receiving sensitivity of external wireless receiver, as well as without the need of attaching the receiving antenna to the patient's waist, is highly desired. The capsule-type endoscopic system with real-time image display according to the present invention can solve the problems described above.

### SUMMARY

The purpose of the present invention is to provide a capsule-type endoscopic system with real-time image display, which displays endoscopic image data produced by a capsule-type endoscope by means of a display apparatus for observing symptoms of digestive diseases without attaching any receiving antenna to the patient's body. Thereby, emergent and dangerous digestive diseases can be treated at early stage, and the effects of medical treatment can be enhanced.

The other purpose of the present invention is to provide a capsule-type endoscopic system with real-time image display, which includes a data recorder for storing endoscopic images extracted by a capsule-type endoscope. Thereby, symptoms in the alimentary canal can be recorded and tracked, and the effects of medical treatment can be enhanced.

The present invention relates to a capsule-type endoscopic system with real-time image display, which includes a capsule-type endoscope, a first wireless receiving unit, and a display apparatus. The capsule-type endoscope includes a camera lens, an image sensor, and a wireless transmitting unit. The first wireless receiving unit couples to the display apparatus. The capsule-type endoscope extracts an endoscopic image inside of a human body by the image sensor via the camera lens, and the image sensor produces endoscopic image data according to the endoscopic image. The wireless transmitting unit transmits the endoscopic image data to the first wireless receiving unit. The display apparatus displays real-timely the endoscopic image data received by the first wireless receiving unit. In addition, the capsule-type endoscopic system according to the present invention further includes a data recorder for storing the endoscopic data produced by the capsule-type endoscope, and for tracking and confirming symptoms in alimentary canals.

### BRIEF DESCRIPTION OF THE DRAWINGS

FIG. 1 is a block diagram of the capsule-type endoscopic system according to the prior art;

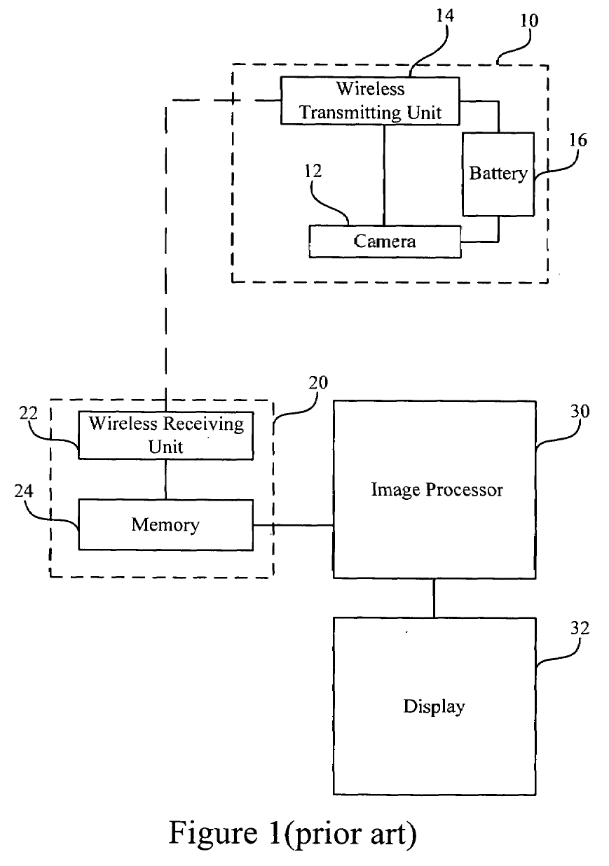

FIG. 2 is a block diagram according to a preferred embodiment of the present invention;

FIG. 3 is a block diagram according to another preferred embodiment of the present invention;

FIG. 4 is a block diagram of the data recorder according to another preferred embodiment of the present invention; and

FIG. 5 is a block diagram according to another preferred embodiment of the present invention.

### DETAILED DESCRIPTION

In order to make the structure and characteristics as well as the effectiveness of the present invention to be further understood and recognized, the detailed description of the present invention is provided as follows along with preferred embodiments and accompanying figures.

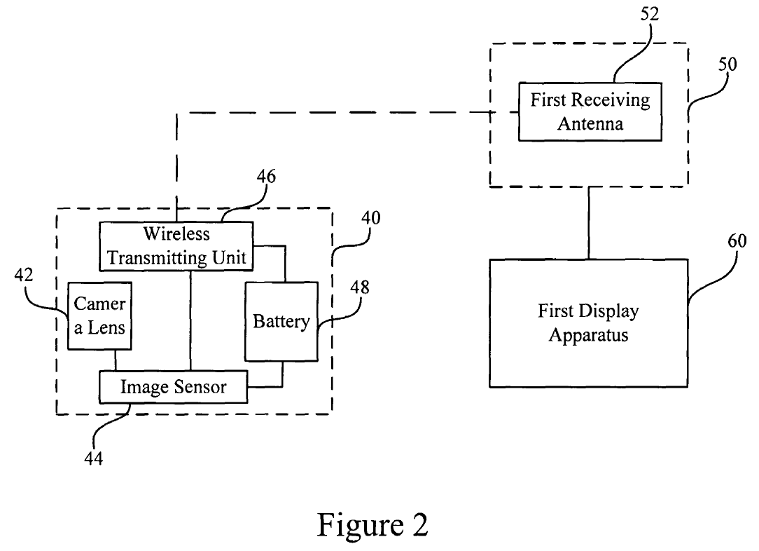

FIG. 2 is a block diagram according to a preferred embodiment of the present invention. As shown in the figure, the capsule-type endoscopic system with real-time image display according to the present invention includes a capsule-type endoscope 40, a first wireless receiving unit 50, and a first display apparatus 60. The capsule-type endoscope 40 includes a camera lens 42, an image sensor 44, a wireless transmitting unit 46, and a battery 48. The wireless transmitting unit 46 adopts the Ultra-High-Frequency (UHF) band. The first wireless receiving unit 50 includes a first receiving antenna 52, which couples to the first display apparatus 60, and also adopts the UHF band corresponding to the band of the wireless transmitting unit 46.

The capsule-type endoscope 40 has the shape and size similar to a medical capsule. The image sensor 44 extracts an endoscopic image through the camera lens 42, and produces endoscopic image data according to the extracted endoscopic image. The endoscopic image data produced by the image sensor 44 is sent to the wireless transmitting unit 46, which transmits the endoscopic image data produced by the image sensor 44 to the first wireless receiving unit 50. The battery 48 supplies power to the image sensor 44 and the wireless transmitting unit 46. In an embodiment of the present invention, the material of the case of the capsule-type endoscope 40 has excellent bio-compatibility;the resolution of the image sensor 44 is 256 by 256 pixels; the image extraction rate of the image sensor 44 is two frames per second (fps). Thereby, patients under endoscopy will not feel like vomiting by swallowing the capsule-type endoscope 40, and hence the capsule-type endoscope 40 can extract image data in the alimentary canal normally.

The first wireless receiving unit 50 receives the endoscopic image data transmitted by the wireless transmitting unit 46 via the first receiving antenna 52, and then sends the received endoscopic image data to the first display apparatus 60 for displaying the endoscopic image data received by the first wireless receiving unit 50 real-timely. In an embodiment of the present invention, the effective receiving range of the first receiving antenna 52 is within 1.5 meters in front of it. Thereby, if the patient under endoscopy is within the effective receiving range of the first receiving antenna 52, the first wireless receiving unit 50 can receive the endoscopic image data transmitted by the wireless transmitting unit 46. Accordingly, when physicians use the capsule-type endoscope according to the present invention to diagnose patients. with digestive diseases, the patients are arranged within the effective receiving range of the first receiving antenna 52 for making the first display apparatus 60 display immediately the endoscopic image of the alimentary canal. If the patients suffer from emergent digestive diseases, the physicians can perform necessary treatments on the patients at earliest stage.

The patients under endoscope do not need to carry the first wireless receiving unit 50 and the first display apparatus 60, therefore it is not necessary to consider their portability and limit their size. In one embodiment of the present invention, the gain of the first receiving antenna 52 according to the present invention is 9 dB to 12 dB, and the first receiving antenna 52 is a directional antenna. No matter where the capsule-type endoscope 40 is in the alimentary canal in a human body, once the patient under endoscopy is within 1.5 meters in front of the first receiving antenna 52, the first wireless receiving unit 50 receives the endoscopic image data via the first receiving antenna 52 transmitted by the wireless transmitting unit 46. Then the first display apparatus 60 displays real-timely the endoscopic image data received by the first wireless receiving unit 50. Besides, the capsule-type endoscopic system with real-time image display of the present invention can further judge if the capsule-type endoscope 40 is normal by the first display apparatus 60 real-timely displaying the endoscopic image data of the alimentary canal of a patient. If the capsule-type endoscope 40 operates abnormally, emergent treatment can be performed immediately.

As described above, the capsule-type endoscopic system with real-time image display according to the present invention provides the first display apparatus 60 for displaying the endoscopic image data produced by the capsule-type endoscope 40 in a human body. Thereby, patients suffering from digestive diseases can be cured at early stage. No matter if the patients' diseases need emergent treatment, they can be discovered and be cured at earliest stage.

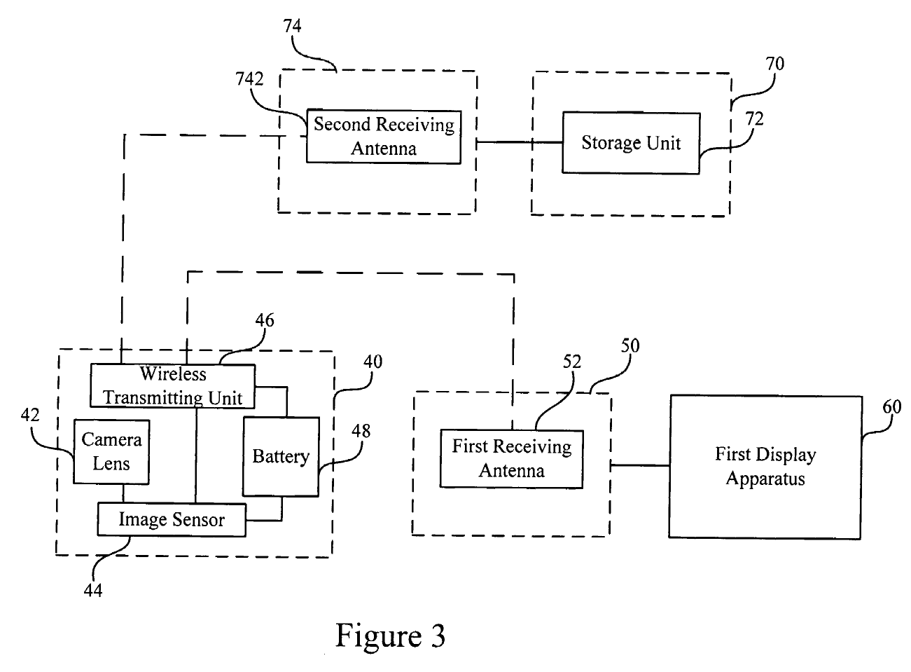

FIG. 3 shows a block diagram according to another preferred embodiment of the present invention. The difference between FIG. 2 and FIG. 3 is that FIG. 3 further includes a data recorder 70. The capsule-type endoscopic system with real-time image display according to the present invention further includes a data recorder 70. The data recorder 70 includes a storage unit 72 and a second wireless receiving unit 74, which includes a second receiving antenna 742 adopting the UHF band. The image sensor 44 extracts an endoscopic image through the camera lens 42, and produces endoscopic image data according to the endoscopic image. The endoscopic image data produced by the image sensor 44 is sent to the wireless transmitting unit 46, which transmits the endoscopic image data produced by the image sensor 44 to the first wireless receiving unit 50 and the second wireless receiving unit 74. The first wireless receiving unit 50 sends the received endoscopic image data to the first display apparatus 60 for displaying real-timely the endoscopic image data received by the first wireless receiving unit 50.

The second wireless receiving unit 74 receives the endoscopic image data transmitted by the wireless transmitting unit 46 via the second receiving antenna 742, and then sends the received endoscopic image data to the storage unit 72 of the data recorder 70 for storing the endoscopic image data. Thereby, the endoscopic images extracted by the capsule-type endoscope 40 can be tracked and confirmed by the physicians as a reference in diagnosis. The receiving sensitivity of the second receiving unit 74 is −80 dBm.

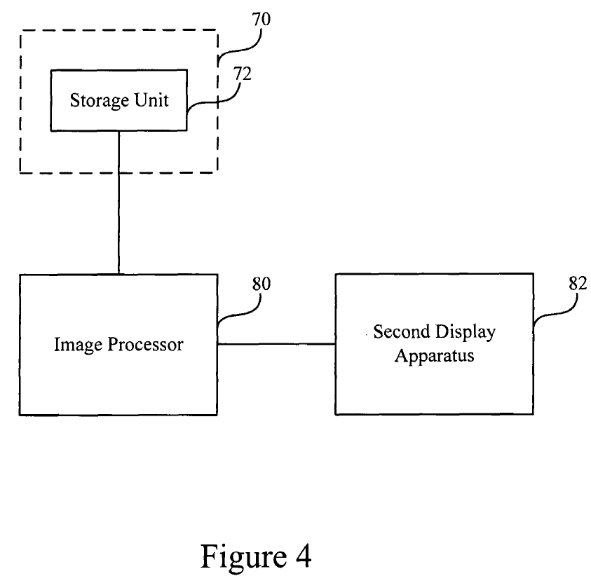

Furthermore, when endoscopy is completed, the data recorder 70 can be disassembled and be coupled to an image processor 80. As shown in FIG. 4, the storage unit 72 of the data recorder 70 is coupled to the image processor 80, which, in turn, is coupled to a second display apparatus 82. The image processor 80 reads the endoscopic image data stored in the storage unit 72, and then sends the read endoscopic image data to the second display apparatus 82. The second display apparatus 82 displays the endoscopic image data read by the image processor 80. Thereby, the examination process of the endoscope is traced and is used for analyzing the nidus. Besides, the second wireless receiving unit 74 of the data recorder 70 can further include the second receiving antenna 742, which is not necessary to be attached to the patients under endoscopy. If the patients under endoscopy are arranged within the effective receiving range of the second wireless receiving antenna 742, the second wireless receiving unit 74 can further receive the endoscopic image data transmitted by the wireless transmitting unit 46 of the capsule-type endoscope 40, and store the endoscopic image data to the data recorder 70.

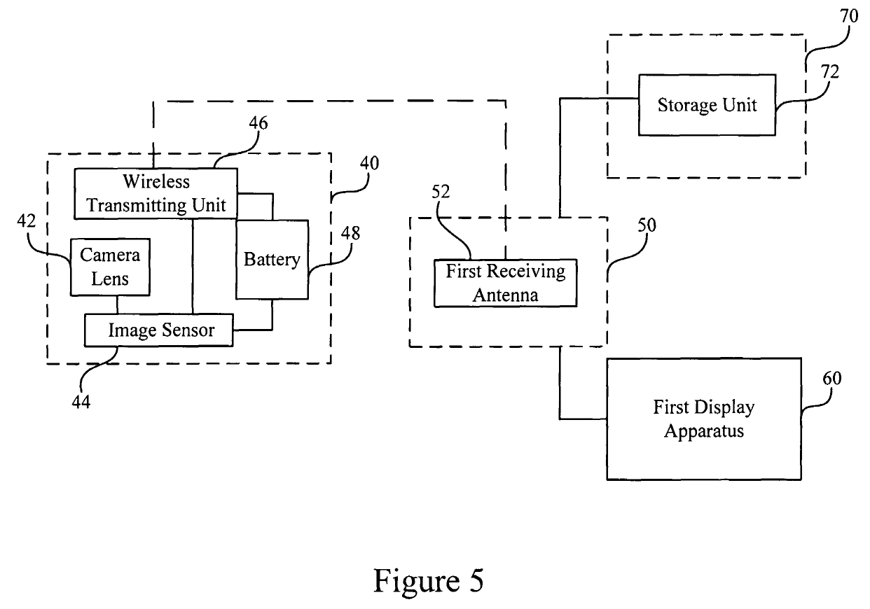

FIG. 5 is a block diagram according to another preferred embodiment of the present invention. The difference between FIG. 4 and FIG. 5 is that the data recorder 70 in FIG. 4 includes the storage unit 72 and the second wireless receiving unit 74, wherein the storage unit 72 is coupled to the second wireless receiving unit 74. However, the data recorder 70 in FIG. 5 includes the storage unit 72 only, wherein the storage unit 72 is coupled to the first wireless receiving unit 50. The capsule-type endoscopic system with real-time image display according to the present invention can further make the first display apparatus 60 and the data recorder 70 both couple to the first wireless receiving unit 50. Thereby, when the first wireless receiving unit 50 receives the endoscopic image data transmitted by the wireless transmitting unit 46 of the capsule-type endoscope 40, the first wireless receiving unit 50 sends immediately the received endoscopic image data to the first display apparatus 60 and the data recorder 70. Hence, the first display apparatus displays real-timely the endoscopic image data received by the first wireless receiving unit 50, and the data recorder 70 stores real-timely and synchronously the endoscopic image data received by the first wireless receiving unit 50.

To sum up, the capsule-type endoscopic system with real-time image display according to the present invention includes a capsule-type endoscope, a first wireless receiving unit, a first display apparatus, and a data recorder. The capsule-type endoscope extracts an endoscopic image in the alimentary canal of a human body, and produces endoscopic image data according to the endoscopic image. Then the capsule-type endoscope transmits the endoscopic image data to the first wireless receiving unit via a wireless transmitting unit for making the first display apparatus display real-timely the endoscopic image data received by the first wireless receiving unit. The data recorder receives the endoscopic image data transmitted by the wireless transmitting unit via a second wireless receiving unit, or via the first wireless receiving unit, for storing the endoscopic image data to a storage unit in the data recorder. Thereby, the examination process can be traced and confirmed after endoscopy.

Accordingly, the present invention conforms to the legal requirements owing to its novelty, unobviousness, and utility. However, the foregoing description is only a preferred embodiment of the present invention, not used to limit the scope and range of the present invention. Those equivalent changes or modifications made according to the shape, structure, feature, or spirit described in the claims of the present invention are included in the appended claims of the present invention.

1. A capsule-type endoscopic system with real-time image display, comprising:
a capsule-type endoscope, comprising a camera lens, an image sensor, and a wireless transmitting unit, the image sensor extracting an endoscopic image through the camera lens, and producing endoscopic image data according to the endoscopic image;
a first wireless receiving unit, receiving the endoscopic image data transmitted by the wireless transmitting unit; and
a first display apparatus, coupling to the first wireless receiving unit, and displaying the endoscopic image data received by the first wireless receiving unit.

2. The capsule-type endoscopic system with real-time image display of claim 1, wherein the capsule-type endoscope further includes a battery, supplying power to the image sensor and the wireless transmitting unit.

3. The capsule-type endoscopic system with real-time image display of claim 1, wherein the first wireless receiving unit further includes a first receiving antenna.

4. The capsule-type endoscopic system with real-time image display of claim 3, wherein the first receiving antenna adopts the Ultra-High-Frequency (UHF) band.

5. The capsule-type endoscopic system with real-time image display of claim 1, and further comprising a data recorder comprising a storage unit and a second wireless receiving unit, the second wireless receiving unit receiving the endoscopic image data transmitted by the wireless transmitting unit, and the storage unit storing the endoscopic image data.

6. The capsule-type endoscopic system with real-time image display of claim 5, wherein the second wireless receiving unit further includes a second receiving antenna.

7. The capsule-type endoscopic system with real-time image display of claim 6, wherein the second receiving antenna adopts the Ultra-High-Frequency (UHF) band.

8. The capsule-type endoscopic system with real-time image display of claim 5, wherein the data recorder further couples to an image processor, which couples to a second display apparatus, the image processor reading the endoscopic image data stored in the storage unit, and outputting the endoscopic image data to the second display apparatus for displaying the endoscopic image data.

9. The capsule-type endoscopic system with real-time image display of claim 1, wherein the first wireless receiving unit further couples to a data recorder comprising a storage unit for storing the endoscopic image data received by the first wireless receiving unit.

10. The capsule-type endoscopic system with real-time image display of claim 9, wherein the data recorder further couples to an image processor coupling to a second display apparatus, the image processor reading the endoscopic image data stored in the storage unit, and outputting the endoscopic image data to the second display apparatus for displaying the endoscopic image data.

11. The capsule-type endoscopic system with real-time image display of claim 1, wherein the wireless transmitting unit adopts the Ultra-High-Frequency (UHF) band.
